(19) United States
(12) Patent Application Publication
(10) Pub. No.: US 2024/0238263 A1
(43) Pub. Date: Jul. 18, 2024

(54) USE OF PYRIDONE DERIVATIVE CONTAINING HETEROATOM CYCLOBUTANE SUBSTITUENT

(71) Applicant: GUANGZHOU JOYO PHARMATECH CO., LTD, Guangzhou (CN)

(72) Inventors: Yongguo LI, Guangzhou (CN); Xiaoning CHEN, Guangzhou (CN); Hongyan MA, Guangzhou (CN)

(73) Assignee: GUANGZHOU JOYO PHARMATECH CO., LTD, Guangzhou, GD (CN)

(21) Appl. No.: 18/562,660

(22) PCT Filed: May 20, 2022
(86) PCT No.: PCT/CN2022/094257

§ 371 Date: Nov. 20, 2023

(30) Foreign Application Priority Data
May 20, 2021 (CN) 202110561093.5
May 6, 2022 (CN) 202210532113.0

(51) Int. Cl. A61K 31/4436; A61K 9/06; A61K 47/10; A61K 47/18; A61K 47/32; A61P 17/02

(52) U.S. Cl. CPC A61K 31/4436; A61K 9/06; A61K 47/10; A61K 47/18; A61K 47/32; A61P 17/02

(57) ABSTRACT

Disclosed is the use of a pyridone derivative containing a heteroatom cyclobutane substituent. Disclosed is the use of substance A in the preparation of a drug. Substance A is a compound as represented by formula I, a pharmaceutically acceptable salt thereof, a solvate thereof, or a solvate of the pharmaceutically acceptable salt thereof. The drug is the drug for treating and/or preventing diabetic feet. The disclosed substance A or the drug containing substance A has good activity on patients with a diabetic foot.

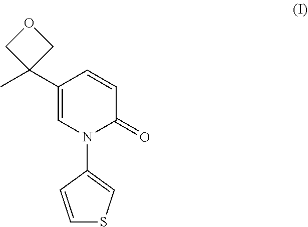 [SMILES: CC1(c2ccc(=O)n(-c3ccsc3)c2)COC1]

The present application claims the right of the priorities of Chinese Patent Application No. 2021105610935, filed on May 20, 2021, and Chinese Patent Application No. 2022105321130, filed on May 6, 2022. The contents of the above Chinese patent applications are incorporated herein by reference in their entireties.

### TECHNICAL FIELD

The present disclosure relates to the technical field of biopharmaceuticals. Specifically, the present disclosure relates to a use of a pyridone derivative containing a heteroatom cyclobutane substituent.

### BACKGROUND

Diabetic foot is a chronic complication associated with diabetes, and is a general term for abnormal changes in the lower extremities caused by diabetic peripheral vascular and nerve damage. Due to concurrent infections, these abnormalities can lead to gangrene of the extremities, which is a significant factor directly placing the patient at risk for disability. Due to its frequent occurrence in the lower extremities, toes, and soles of the feet, it is clinically referred to as diabetic foot. The primary cause is prolonged exposure to a high blood glucose state without optimal control, leading to pathological changes in both the macrovascular and microvascular systems of the lower extremities. This subsequently results in the extremities falling into a state of ischemia and hypoxia, causing the tissues to lose their vitality.

The wound healing process for diabetic foot is a complex, multi-stage process. Due to various complexities, there is insufficient angiogenesis and re-epithelialization in the patient. Severe inflammation, caused by neutrophil infiltration, is another detrimental factor. Additionally, diabetic foot is the result of severe inflammation. Driven by the increased risk of developing diabetic foot among diabetes patients, there is a decline in quality of life and a loss of productivity, which also imposes a significant economic burden on the healthcare system. The direct and indirect annual medical costs for Type 2 diabetes patients with diabetic foot are estimated to be around $13,561. Evidence suggests that various factors contributing to delayed wound healing include elevated blood glucose levels, increased insulin resistance, reduced angiogenesis and collagen deposition, delayed infiltrative response, obstructed blood supply to the wound site, decreased granulation tissue formation, and increased blood viscosity. Despite significant breakthroughs in the research on diabetic foot over the past few decades, effectively treating diabetic foot remains a challenge.

### CONTENT OF THE PRESENT INVENTION

The technical problem to be solved by the present disclosure is the relatively limited structural diversity of existing drugs for treating diabetic foot. The present disclosure provides a use of a pyridone derivative containing a heteroatom cyclobutane substituent. The substance and a medicament containing the substance are able to promote wound healing in diabetic foot.

The present disclosure provides a use of a substance A in the manufacture of a medicament,

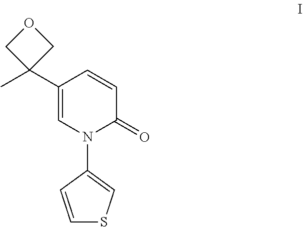 [SMILES: CC1(c2ccc(=O)n(-c3ccsc3)c2)COC1]

the medicament is a medicament for treating and/or preventing diabetic foot;
the substance A is a compound of formula I, a pharmaceutically acceptable salt thereof, a solvate thereof, or a solvate of the pharmaceutically acceptable salt thereof.

In the use, the medicament treats and/or prevents diabetic foot by enhancing wound-healing ability of patients with diabetic foot.

In the use, the medicament comprises (i) the substance A and (ii) a pharmaceutically acceptable carrier.

In the use, the medicament has a dosage form of an external preparation, such as a cream, an ointment, or a gel, preferably a gel.

In the use, the medicament may be administered at a frequency of once a day, twice a day, or three times a day. Each time the medicament is administered, the dosage of the substance A contained in the medicament can be determined based on the actual therapeutic effect observed in the test subject or patient. Preferably, in the use, the compound of formula I in the substance A accounts for 0.1% to 4% of the medicament by mass, such as 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%.

In the use, the compound of formula I has a mass fraction of 0.3% to 3%, such as 0.3%, 1%, or 3%.

In the use, the medicament consists of 0.3% to 3% of the compound of formula I, 1% Carbopol 940NF, 50% propylene glycol, 13% diethylene glycol monoethyl ether, 1% triethanolamine, and water, with water making up the remainder to 100%.

The present disclosure also provides a method for treating and/or preventing diabetic foot, the method comprising administering a therapeutically effective amount of the substance A or the medicament to a patient; the medicament comprises (i) the substance A and (ii) a pharmaceutically acceptable carrier; the substance A is a compound of formula I, a pharmaceutically acceptable salt thereof, a solvate thereof, or a solvate of the pharmaceutically acceptable salt thereof;

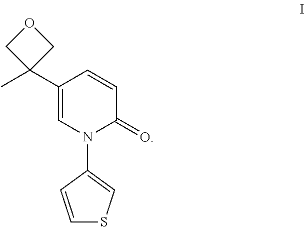 [SMILES: CC1(c2ccc(=O)n(-c3ccsc3)c2)COC1]

In the method for treating and/or preventing diabetic foot, the substance A or the medicament may be administered at a frequency of once a day, twice a day, or three times a day. Each time the medicament is administered, the dosage of substance A contained in the medicament can be determined based on the actual therapeutic effect observed in the test subject or patient. Preferably, the compound of formula I in the substance A accounts for 0.1% to 4% of the medicament by mass, such as 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%.

In the method for treating and/or preventing diabetic foot, the compound of formula I has a mass fraction of 0.3% to 3%, such as 0.3%, 1%, or 3%.

In the method for treating and/or preventing diabetic foot, the medicament consists of 0.3% to 3% of the compound of formula I, 1% Carbopol 940NF, 50% propylene glycol, 13% diethylene glycol monoethyl ether, 1% triethanolamine, and water, with water making up the remainder to 100%.

In the method for treating and/or preventing diabetic foot, the compound of formula I or the medicament treats and/or prevents diabetic foot by enhancing wound-healing ability of patients with diabetic foot.

In the method for treating and/or preventing diabetic foot, the dosage form of the medicament is an external preparation, such as a cream, an ointment, or a gel, preferably a gel.

The present disclosure also provides a pharmaceutical composition, which comprises a substance A and a pharmaceutically acceptable carrier; the substance A is a compound of formula I, a pharmaceutically acceptable salt thereof, a solvate thereof, or a solvate of the pharmaceutically acceptable salt thereof; the compound of formula I has a mass fraction of 0.1% to 4%;

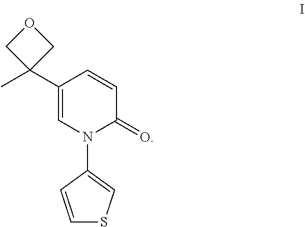 [SMILES: CC1(c2ccc(=O)n(-c3ccsc3)c2)COC1]

In the composition, preferably, the compound of formula I has a mass fraction of 0.3% to 3%, such as 0.3%, 1%, or 3%.

The composition consists of 0.3% to 3% of the compound of formula I, 1% Carbopol 940NF, 50% propylene glycol, 13% diethylene glycol monoethyl ether, 1% triethanolamine, and water, with water making up the remainder to 100%.

In the composition, preferably, the composition is used for treating and/or preventing diabetic foot.

The present disclosure also provides an external composition, which comprises a substance A and a pharmaceutically acceptable carrier; the substance A is a compound of formula I, a pharmaceutically acceptable salt thereof, a solvate thereof, or a solvate of the pharmaceutically acceptable salt thereof;

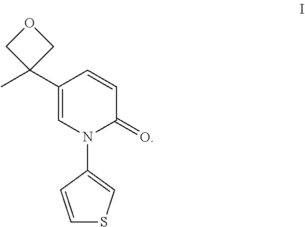 [SMILES: CC1(c2ccc(=O)n(-c3ccsc3)c2)COC1]

In the external composition, preferably, the compound of formula I has a mass fraction of 0.1% to 4%; preferably, the compound of formula I has a mass fraction of 0.3% to 3%, such as 0.3%, 1%, or 3%.

In the external composition, the pharmaceutically acceptable carrier is a conventional carrier commonly used in such external compositions in the art; preferably, the pharmaceutically acceptable carrier comprises at least Carbopol, propylene glycol, diethylene glycol monoethyl ether, triethanolamine, and water.

In the present disclosure, the Carbopol is selected from one or more than one of Carbopol 940NF, Carbopol 934, Carbopol 974, Carbopol 981, or Carbopol 1342.

In the external composition, the external composition may have a dosage form of a cream, an ointment, or a gel, such as a gel.

In the external composition, preferably, the external composition can be used for treating and/or preventing diabetic foot.

The external composition consists of 0.3% to 3% of the compound of formula I, 1% Carbopol 940NF, 50% propylene glycol, 13% diethylene glycol monoethyl ether, 1% triethanolamine, and water, with water making up the remainder to 100%. The present disclosure also provides a gel, which comprises a substance A and a pharmaceutically acceptable carrier; the substance A is a compound of formula I, a pharmaceutically acceptable salt thereof, a solvate thereof, or a solvate of the pharmaceutically acceptable salt thereof;

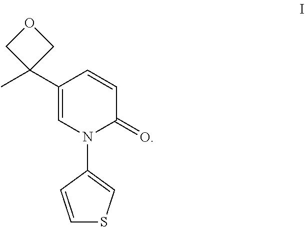 [SMILES: CC1(c2ccc(=O)n(-c3ccsc3)c2)COC1]

In the gel, preferably, the compound of formula I has a mass fraction of 0.1% to 4%; preferably, the compound of formula I has a mass fraction of 0.3% to 3%, such as 0.3%, 1%, or 3%.

In the gel, the pharmaceutically acceptable carrier is a conventional carrier commonly used in such gel in the art; preferably, the pharmaceutically acceptable carrier is Carbopol, propylene glycol, diethylene glycol monoethyl ether, triethanolamine, and water.

In the gel, calculated by mass percentage, the gel comprises 0.3% to 3% of the compound of formula I, 1% of the Carbopol 940NF, 50% of the propylene glycol, 13% of the diethylene glycol monoethyl ether, 1% of the triethanolamine, with water making up the remainder to 100%.

In the gel, preferably, the external composition can be used for treating and/or preventing diabetic foot.

The gel consists of 0.3% to 3% of the compound of formula I, 1% Carbopol 940NF, 50% propylene glycol, 13% diethylene glycol monoethyl ether, 1% triethanolamine, and water, with water making up the remainder to 100%.

In the present disclosure, the mass fraction of the compound of formula I refers to the mass corresponding to the structure of the compound of formula I relative to the total mass of the medicament or composition. For example, when the compound of formula I exists in the form of a salt, the mass ratio is calculated based on its anion.

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable carrier” refers to any formulation or carrier medium that can deliver an effective amount of the active substance of the present disclosure, does not interfere with the biological activity of the active substance, and has no toxicity or side effects on the host or patient. Representative carriers include water, oils, vegetables, minerals, paste bases, lotion bases, ointment bases, and the like. These bases include suspending agents, viscosity-increasing agents, transdermal enhancers, etc. Their formulations are well known to those skilled in the field of cosmetics or topical medication. For additional information on carriers, one can refer to “Remington: The Science and Practice of Pharmacy, 21st Ed., Lippincott, Williams & Wilkins (2005),” the contents of which are incorporated herein by reference.

The term “treatment” refers to therapeutic therapy. When referring to a specific disorder, treatment refers to: (1) ameliorating one or more biological manifestations of the disease or disorder, (2) interfering with (a) one or more points in the biological cascade leading to or causing the disorder or (b) one or more biological manifestations of the disorder, (3) ameliorating one or more symptoms, effects, or side effects associated with the disorder, or one or more symptoms, effects or side effects associated with the disorder or its treatment, or (4) slowing the progression of the disorder or one or more biological manifestations of the disorder.

The term “prevention” refers to the reduction of the risk of acquiring or developing diseases or disorders.

The term “therapeutically effective amount” refers to the quantity of the compound that, when administered to a patient, is sufficient to effectively treat the disease or condition described herein. The term “therapeutically effective amount” will vary based on the compound, the disease and its severity, as well as the age of the patient to be treated, but can be adjusted as needed by those skilled in the art.

The term “patient” refers to any animal that will receive, or has already received, administration of the compound according to examples of the present disclosure, with mammals being preferred and humans being most preferred. The term “mammal” includes any mammal. Examples of mammals include but are not limited to cows, horses, sheep, pigs, cats, dogs, mice, rats, rabbits, guinea pigs, monkeys, and humans, with humans being the most preferred.

Without violating common knowledge in the art, the aforementioned preferred conditions can be arbitrarily combined to yield the various preferred embodiments of the present disclosure.

The reagents and raw materials used in the present disclosure are all commercially available.

The positive progressive effect of the present disclosure lies in that: the present disclosure provides a use of a substance A in the manufacture of a medicament, the substance A being the compound of formula I, the pharmaceutically acceptable salt thereof, the solvate thereof, or the pharmaceutically acceptable salt thereof. The substance A or the medicament containing the substance A in the present disclosure demonstrates good activity in patients with diabetic foot.

### BRIEF DESCRIPTION OF THE DRAWINGS

FIG. 1 shows effects of topical administration of compound I gel on fasting blood glucose in diabetic rats;

FIG. 2 shows effects of topical administration of compound I for 11 days on remaining open wound area % in diabetic rats;

FIG. 3 shows representative photographs of wound healing on day 1 and day 11 for each experimental group.

### DETAILED DESCRIPTION OF THE PREFERRED EMBODIMENT

The present disclosure will be further illustrated by way of examples below, but it is not limited to the scope of these examples. In the following examples, experimental methods not specified with particular conditions are carried out using conventional methods and conditions, or are selected according to the product instructions.
1. The compound I referred to in the following examples is

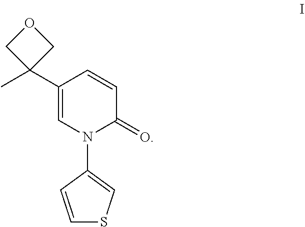 [SMILES: CC1(c2ccc(=O)n(-c3ccsc3)c2)COC1]

2. Experimental materials:
1) Test substances: Compound I is prepared into gels containing, respectively, a mass fraction of 0.3% of compound I, a mass fraction of 1% of compound I, and a mass fraction of 3% of compound I. Taking the gel with a mass fraction of 3% of compound I as an example, the mass fraction of each component is shown in Table 1 below:

TABLE 1

| Proportion of raw and auxiliary materials | (w/w) % |
|---|---|
| Compound I | 3.0 |
| Carbopol 940NF | 1.0 |
| Propylene glycol | 50.0 |
| Diethylene glycol monoethyl ether | 13.0 |
| Triethanolamine | 1.0 |
| Water | 32.0 |
| Total | 100.0 |

The gels with 0.3% and 1% mass fractions of compound I differ from the gel with a 3% mass fraction of compound I only in the mass fraction of compound I. The complete ratio is adjusted to 100% with water to make up for the difference.

Supplier: Compound I is provided by GUANGZHOU JOYO PHARMATECH CO., LTD., and can also be prepared according to example 2 in WO2017177974A1.

Administration mode: 50 mg is directly administered to each wound.
2) Positive control:

GeneTime (Recombinant Human Epidermal Growth Factor External Solution) Specifications: 2000 IU/mL, 15 mL/bottle

Supplier: Zhuhai Essex Bio-Technology Limited.

### Example 1: Pharmacological Evaluation in a Dorsal Skin Full-Thickness Excisional Wound Model in STZ-Induced Diabetic Rats

1. Animals: Wistar rats, male, 15-16 weeks old, weighing approximately 220 g; Supplier: Shanghai SIPPR-BK Lab Animal Co., Ltd.;

In this study, 50 STZ-diabetic rats were used to simulate Type 1 diabetes, with an additional 8 normal rats serving as the control group (normal blank gel control group (without STZ)). The method for modeling the STZ-diabetic rat is as follows: Male Wistar rats were weighed and then intraperitoneally injected with Streptozotocin (STZ, freshly prepared, dissolved in 0.1 M citrate buffer (pH 4.5)) at a dosage of 80 mg/kg. STZ was intraperitoneally injected twice in total, with a 5-day interval between injections. Three days after the second STZ injection, rats with fasting blood glucose levels greater than 250 mg/dl were defined as diabetic model rats. Studies on skin wound healing could be conducted from 1 to 8 weeks after the successful establishment of the diabetic rat model.
2. The dosing groups and dosing regimens are as shown in Table 2.

TABLE 2

| | Number | | | | |
|---|---|---|---|---|---|
| Group | of rats | Test substance | Dosage | | Dosing schedule^{a} |
| G1: Model blank gel control group | 10 | Blank gel | 50 | mg gel | Twice daily, continuous dosing for 18 days |
| G2: Model 0.3% compound I treatment group | 10 | 0.3% Compound I | 50 | mg gel | Twice daily, continuous dosing for 18 days |
| G3: Model 1% compound I treatment group | 10 | 1% Compound I | | | Twice daily, continuous dosing for 18 days |
| G4: Model 3% compound I treatment group | 10 | 3% Compound I | | | Twice daily, continuous dosing for 18 days |
| G5: Model GeneTime control group | 10 | GeneTime | 0.04 | mL | Twice daily, continuous dosing for 18 days |
| G6: Normal blank gel control group (without STZ) | 8 | Blank gel | 50 | mg gel | Twice daily, continuous dosing for 18 days |

^{a}If there were animals with wounds that have not healed, the administration would be extended until the wounds heal.

3. Preparation of dorsal skin full-thickness excisional wound model

In diabetic model rats, the dorsal skin was marked with a stamp, and full-thickness excision was performed to create a standardized wound area. The day of the full-thickness skin excision surgery was designated as Day 1 of the experiment. The specified surgical procedure was as follows:
1) Prior to surgery, animals were anesthetized with 2-5% isoflurane. The back was shaved and prepared, and the surgical area was disinfected with iodophor, followed by de-iodination with 75% alcohol.
2) All animals were marked on the skin of their backs using a stamp with a circular pattern. A circular full-thickness skin excision was performed along the midline of the rat's back, extending down to the fascial layer. Each incision had a diameter of approximately 1.5 cm. The surgical site was essentially consistent for each animal;
3) Post-surgery, every 8-12 hours, 8 mg/kg of bupivacaine (diluted to 0.25% with physiological saline) was injected subcutaneously at the wound site. All animals were closely monitored until they regained consciousness. Animals were monitored daily, and if symptoms of pain persisted, they would continue to receive pain medication.
4. Drug administration:

All experimental groups were topically administered. Before administering the medication, wounds were rinsed with physiological saline and any residual moisture was absorbed with a sterile cotton swab. Then, the medication was applied. For the GeneTime group, the solution was evenly applied to the wound and surrounding skin, with the dressing changed twice a day. For the other groups, the gel samples were applied from the periphery toward the center, focusing first on the edges of the lesion before covering inward, with the dressing changed twice a day. Medication was applied immediately after the wound model was created, and each group was continuously administered for 18 days. For specific administration information, see Table 2.
5. Efficacy assessment
1) Blood glucose monitoring: Blood samples were obtained from all animals via tail vein sampling, and fasting blood glucose levels were measured on days 0/9/18 using a glucometer.
2) Evaluation of wound closure:

After anesthetizing the rats with isoflurane, a ruler was placed near the wound as a reference scale. Using a digital camera, photos of the wound on the rat's back were taken on days 1, 4, 8, 11, 14, and 18, maintaining a constant focus and pixel condition. The obtained images were processed by software to measure the wound area.
6. Statistical analysis

Experimental data were expressed as the mean±standard error of the mean (mean±S.E.M.). Statistical analyses were conducted using appropriate methods in software such as Graphpad Prism or SPSS. A p-value of less than 0.05 is considered to indicate a significant difference.
7. Results
1) Fasting blood glucose

TABLE 3
Effects of topical administration of test compound I on fasting blood glucose levels in diabetic rats

| | Fasting blood glucose (mg/dl) | | |
|---|---|---|---|
| Group | Day 0 | Day 9 | Day 18 |
| G1: Model blank gel control group | 445.68 ± 7.17*** | 429.48 ± 14.96*** | 456.48 ± 14.89*** |
| G2: Model 0.3% compound I treatment group | 431.64 ± 20.81 | 449.64 ± 7.22 | 433.62 ± 5.81 |
| G3: Model 1% compound I treatment group | 432.9 ± 9.21 | 447.3 ± 10.77 | 474.3 ± 7.32 |
| G4: Model 3% compound I treatment group | 418.86 ± 12.33 | 428.22 ± 13.68 | 465.66 ± 12.26 |
| G5: Model GeneTime control group | 432.54 ± 12.9 | 451.8 ± 11.33 | 464.04 ± 3.71 |
| G6: Normal blank gel control group | 85.5 ± 1.86 | 83.93 ± 1.63 | 90.9 ± 1.67 |

Note:
***p < 0.001, compared to the normal blank gel control group.

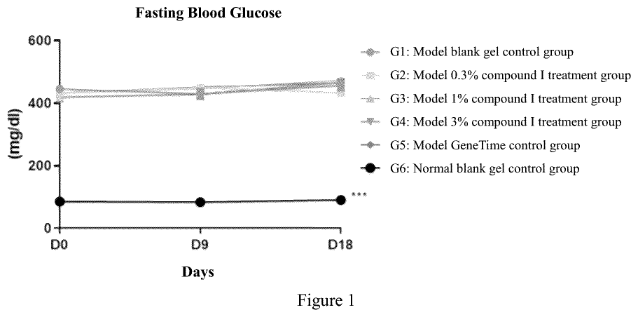

During the course of the experiment, fasting blood glucose was detected on days 0, 9, and 18. As indicated in FIG. 1 and Table 3, when compared to the normal blank gel control group (G6), all the diabetic model groups (G1-5) exhibited a notable increase in blood glucose levels, which were consistently maintained above 400 mg/dL during the experiment. Compared with the model blank gel control group G1, the drug treatment of each drug group has no significant effect on the blood glucose of animals.
2) Remaining open wound area %

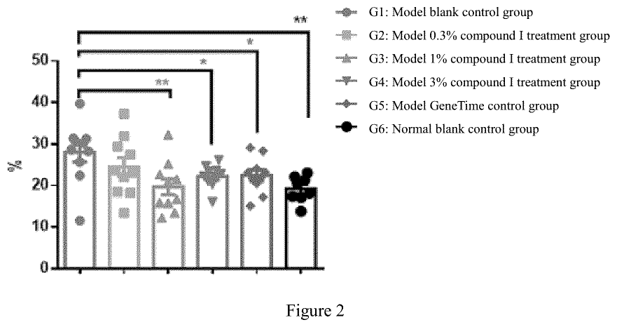

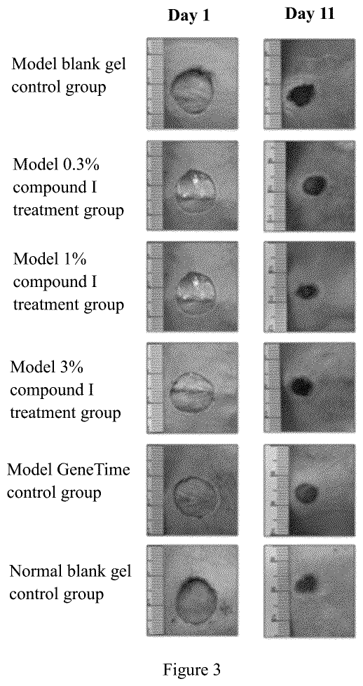

Treatment and administration in this experiment commenced on the day of surgery, applying the drug to the wound surface twice daily until day 18 post-surgery or until the wound was completely healed. 11 days after topical administration, the remaining open wound area % in the reference drug GeneTime group was significantly lower than that in the model blank gel control group (22.43%±1.37% vs. 27.99%±2.31%, P<0.05), suggesting that GeneTime promotes the healing of diabetic wounds. In comparison to the model blank gel control group, both the 1% and 3% compound I groups also significantly reduced the remaining open wound area % on day 11 of administration (19.66%±1.93% vs. 27.99%±2.31%, P<0.01; 22.23%±0.87% vs. 27.99%±2.31%, P<0.05), indicating that compound I promotes diabetic wound healing at both 1% and 3% concentrations (Table 4 and FIG. 2). After 14 days of topical administration, it was observed that the remaining open wound area % tended to decrease under the 1% and 3% concentration levels of test compound I gel, but there was no statistical difference (Table 4). Representative photographs depicting the healing of wounds on day 1 and day 11 for each experimental group are shown in FIG. 3.

TABLE 4
Effects of topical administration of test compound I
on the remaining open wound area % in diabetic rats

| | Remaining open wound area (%) | | | | | |
|---|---|---|---|---|---|---|
| Group | Day 1 | Day 4 | Day 8 | Day 11 | Day 14 | Day 18 |
| Model blank | 100% ± | 92.96% ± | 58.52% ± | 27.99% ± | 8.72% ± | 1.59% ± |
| gel control | 0% | 3.08% | 2.74% | 2.31% | 1.11% | 0.47% |
| group | | | | | | |
| Model 0.3% | 100% ± | 94.82% ± | 54% ± | 24.5% ± | 7.75% ± | 1.22% ± |
| compound I | 0% | 2.58% | 2.22% | 2.25% | 1.86% | 0.54% |
| treatment | | | | | | |
| group | | | | | | |
| Model 1% | 100% ± | 94.25% ± | 55.4% ± | 19.66% ± | 5.47% ± | 0.78% ± |
| compound I | 0% | 3.09% | 3.29% | 1.93%** | 0.86% | 0.35% |
| treatment | | | | | | |
| group | | | | | | |
| Model 3% | 100% ± | 93.96% ± | 56.92% ± | 22.23% ± | 6% ± | 2.01% ± |
| compound I | 0% | 2.67% | 1.98% | 0.87%* | 1.04% | 0.52% |
| treatment | | | | | | |
| group | | | | | | |
| Model | 100% ± | 91.82% ± | 52.36% ± | 22.43% ± | 8.17% ± | 1.79% ± |
| GeneTime | 0% | 3.18% | 2.84% | 1.37%* | 0.92% | 0.73% |
| control group | | | | | | |
| Normal blank | 100% ± | 101.13% ± | 54.21% ± | 19.21% ± | 4.26% ± | 0.85% ± |
| gel control | 0% | 3.51% | 3.58% | 1.09%** | 0.61%* | 0.33% |
| group | | | | | | |

Note:
*indicates p < 0.05,
**indicates p < 0.01, compared with the blank gel control group with the diabetic model.

Remaining Open Wound Area %=Open Wound Area (O)/Initial Wound Area (I)*100%

Conclusion: After inducing diabetes in rats using STZ, a dorsal skin full-thickness excisional wound model was developed. Test substances were administered topically twice a day for a continuous 18-day period. Compared to the model blank gel control group, both 1% and 3% concentrations of compound I gel, as well as the control GeneTime, significantly reduced the remaining open wound area % on day 11 after administration. These results indicate that both the 1% and 3% concentrations of compound I promote wound healing.

While the foregoing describes specific embodiments of the present disclosure, it should be understood by those skilled in the art that these are provided by way of example only. Various modifications or changes can be made to these embodiments without departing from the principles and essence of the present disclosure. Therefore, the scope of protection for the present disclosure is defined by the appended claims.

1-3. (canceled)

4. A method for treating and/or preventing diabetic foot, wherein the method comprises administering a therapeutically effective amount of a substance A or a medicament to a patient; the medicament comprises (i) the substance A and (ii) a pharmaceutically acceptable carrier; the substance A is a compound of formula I, a pharmaceutically acceptable salt thereof, a solvate thereof, or a solvate of the pharmaceutically acceptable salt thereof;

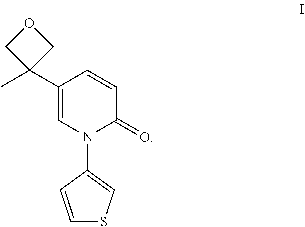 [SMILES: CC1(c2ccc(=O)n(-c3ccsc3)c2)COC1]

5. The method for treating and/or preventing diabetic foot according to claim 4, wherein the substance A or the medicament is administered at a frequency of once a day, twice a day, or three times a day;
or, the substance A or the medicament treats and/or prevents diabetic foot by enhancing wound-healing ability of patients with diabetic foot;
or, the medicament has a dosage form of an external preparation;
or, the compound of formula I in the substance A accounts for 0.1% to 4% of the medicament by mass.

6. The method for treating and/or preventing diabetic foot according to claim 5, wherein the dosage form of the medicament is a cream, an ointment, or a gel;
or, the compound of formula I in the substance A accounts for 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0% of the medicament by mass.

7. A pharmaceutical composition, wherein the pharmaceutical composition comprises a substance A and a pharmaceutically acceptable carrier; the substance A is a compound of formula I, a pharmaceutically acceptable salt thereof, a solvate thereof, or a solvate of the pharmaceutically acceptable salt thereof; the compound of formula I has a mass fraction of 0.1% to 4%;

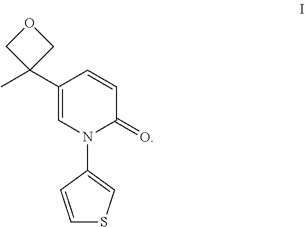 [SMILES: CC1(c2ccc(=O)n(-c3ccsc3)c2)COC1]

8. An external composition, wherein the external composition comprises a substance A and a pharmaceutically acceptable carrier; the substance A is a compound of formula I, a pharmaceutically acceptable salt thereof, a solvate thereof, or a solvate of the pharmaceutically acceptable salt thereof;

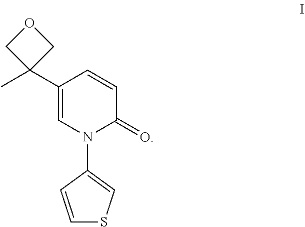 [SMILES: CC1(c2ccc(=O)n(-c3ccsc3)c2)COC1]

9. A gel, wherein the gel comprises a substance A and a pharmaceutically acceptable carrier; the substance A is 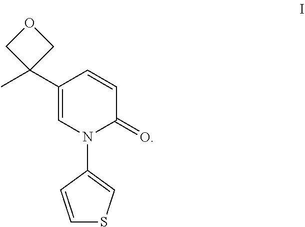 [SMILES: CC1(c2ccc(=O)n(-c3ccsc3)c2)COC1]
as defined in claim 4.

10. The gel according to claim 9, wherein the gel, calculated by mass percentage, comprises 0.3% to 3% of the compound of formula I, 1% of Carbopol 940NF, 50% of the propylene glycol, 13% of the diethylene glycol monoethyl ether, 1% of the triethanolamine, with water making up the remainder to 100%.

11. The method for treating and/or preventing diabetic foot according to claim 6, wherein the dosage form of the medicament is a gel.

12. The pharmaceutical composition according to claim 7, wherein the compound of formula I has a mass fraction of 0.3% to 3%.

13. The pharmaceutical composition according to claim 12, wherein the compound of formula I has a mass fraction of 0.3%, 1%, or 3%.

14. A The external composition according to claim 8, wherein the compound of formula I has a mass fraction of 0.1% to 4%;
or, the pharmaceutically acceptable carrier comprises at least Carbopol, propylene glycol, diethylene glycol monoethyl ether, triethanolamine, and water;
or, in the external composition, the external composition has a dosage form of a cream, an ointment, or a gel.

15. The external composition according to claim 14, wherein the compound of formula I has a mass fraction of 0.3% to 3%;
or, the Carbopol is selected from one or more than one of Carbopol 940NF, Carbopol 934, Carbopol 974, Carbopol 981, or Carbopol 1342;
or, in the external composition, the external composition has a dosage form of a gel.

16. The external composition according to claim 15, wherein the compound of formula I has a mass fraction of 0.3%, 1%, or 3%.

17. The gel according to claim 9, wherein the compound of formula I has a mass fraction of 0.1% to 4%;
or, the gel is an external preparation;
or, the pharmaceutically acceptable carrier comprises at least Carbopol, propylene glycol, diethylene glycol monoethyl ether, triethanolamine, and water.

18. The gel according to claim 17, wherein the compound of formula I has a mass fraction of 0.3% to 3%.

19. The gel according to claim 18, wherein the compound of formula I has a mass fraction of 0.3%, 1%, or 3%.
